(19) United States
(12) Patent Application Publication
(10) Pub. No.: US 2009/0238815 A1
(43) Pub. Date: Sep. 24, 2009

(54) Nondegradable Hydrogels For Medical Device Application

(75) Inventors: Kishore Udipi, Santa Rosa, CA (US); Julie Trudel, Santa Rosa, CA (US); Ya Guo, Rohnert Park, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/050,309

(22) Filed: Mar. 18, 2008

(51) Int. Cl. A61K 39/395; A61K 33/00; C08F 118/00; C08F 116/12; A61P 41/00

(52) U.S. Cl. 424/130.1; 424/718; 526/319; 526/332; 514/44 R; 424/422

(57) ABSTRACT

Disclosed are nondegradable hydrogels used to coat or form at least a portion of a medical device. The hydrogels may be formed from functionalized PEG based macromers. These hydrogels are easy to sterilize, transport and store. Methods of forming these hydrogels as described herein are included.

### FIELD OF THE INVENTION

The present invention relates to non-degradable hydrogels, their use in medical devices and methods for forming the hydrogels.

### BACKGROUND OF THE INVENTION

Polymers often contain matrices within their macrostructure networks. One type of polymeric matrix is a hydrogel, which can be defined as a water-containing polymeric network. Hydrogels have been beneficially used in medical settings for, for example, bioactive agent delivery, prevention of post-surgical adhesions, tissue repair, etceteras.

Hydrogels are often biodegradable, meaning that the polymer, when implanted into a living being will be broken down, the rate of which can be controlled by the physical properties of the hydrogel; biodegradable hydrogels are difficult to sterilize and store as a result of their delicate nature. Nondegradable hydrogels, however, have increased tolerance to the sterilization process and can be stored for significantly increased periods of time without degradation.

Nondegradable hydrogels can be coated onto medical devices or even form portions of medical devices. If the medical device is formed from a hydrogel and the device is to be permanent, for example, a nondegradable hydrogel is essential. Coating specific regions of medical devices can also be important as, in such a case, a specific property of the hydrogel can be imparted at specific locations on the medical device.

Although there are a variety of methods for producing hydrogels, when these networks are intended to be created for presence in or near viable tissue, and/or to contain a bioactive agent, and/or to be created on viable tissue, the number of acceptable methods for producing them becomes limited. For example, one method to produce a hydrogel includes solvent casting of hydrophobic polymers. Solvent casting, however, typically involves the use of organic solvents and/or high temperatures which can be detrimental to the activity of bioactive agents and can complicate production methods. Solvent casting of polymers out of solution also results in the formation of non-cross-linked matrices. Non-cross-linked matrices have less structure than cross-linked matrices and, as a result, it can be more difficult to control the release of bioactive agents from such matrices.

U.S. Pat. No. 5,410,016 (Hubbell, et al.) and U.S. Pat. No. 5,529,914 (Hubbell, et al.) relate to the preparation of hydrogels from biodegradable and biostable polymerizable macromers. The hydrogels are prepared from these polymerizable macromers by the use of soluble, low molecular weight initiators. U.S. Pat. No. 5,232,984 (Hubbell, et al.), U.S. Pat. No. 5,380,536 (Hubbell, et al.), U.S. Pat. No. 5,573,934 (Hubbell, et al.), U.S. Pat. No. 5,612,050 (Rowe, et al.), U.S. Pat. No. 5,837,747 (Soon-Shiong, et al.), U.S. Pat. No. 5,846,530 (Soon-Shiong, et al.), and U.S. Pat. No. 5,858,746 (Hubbell, et al.) also describe various methods of forming hydrogels.

Hydrogels formed using such methods, however, often have limited adhesion to tissue. Therefore, additional methods of forming tissue-adhesive hydrogels are needed.

### SUMMARY OF THE INVENTION

Disclosed herein are polyethylene glycol (PEG) based hydrogels which can be coated on at least a portion of a medical device. The polyethylene glycol (PEG) based hydrogels described herein are also suitable for formation of at least a portion of a medical device. The hydrogels may be tailored to possess adhesive properties, making them useful in the field of implantable medical devices.

In one embodiment, a polymeric material is described, formed from macromers of a general formula 1:

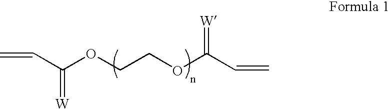

wherein W, W′ are each independently O or CH₂, n is between about 1 and about 500, macromers of a general formula 2:

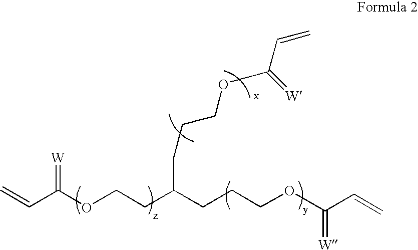

wherein W, W′ and W″ are each independently O or CH₂, x, y, and z are each independently between about 1 and about 500, or combinations thereof, and wherein at least two macromers are cross-linked thereby forming a hydrogel. In another embodiment, the hydrogel is non-degradable. In one embodiment, the hydrogel comprises any ratio of formula 1: formula 2. In one embodiment, at least one of n, x, y, or z is the following:

n is between 1 and 250;

x is between 1 and 250;

y is between 1 and 250; and

z is between 1 and 250.

In one embodiment, the hydrogel further comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. In one embodiment, the hydrogel further comprises at least one bioactive agent selected from the group consisting of sirolimus (rapamycin), tacrolimus (FK506), everolimus (certican), temsirolimus (CCI-779) and zotarolimus (ABT-578).

In one embodiment, an implantable medical device is described comprising a polymeric material, said polymeric material formed from macromers of a general formula 1:

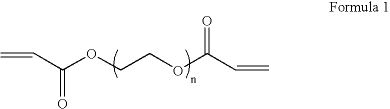

wherein n is between about 1 and about 500, macromers of a general formula 2:

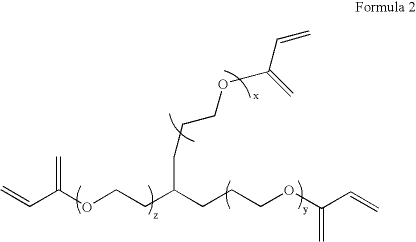

wherein x, y, and z are each independently between about 1 and about 500, or combinations thereof, and wherein at least two macromers are cross-linked thereby forming a hydrogel. In one embodiment, the hydrogel is non-degradable. In one embodiment, the hydrogel comprises any ratio of formula 1: formula 2. In another embodiment, the hydrogel comprises at least one of n, x, y, or z with the following:

n is between 1 and 250;

x is between 1 and 250;

y is between 1 and 250; and

z is between 1 and 250.

In one embodiment, the polymeric material further comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. In another embodiment, the hydrogel further comprises at least one bioactive agent selected from the group consisting of sirolimus (rapamycin), tacrolimus (FK506), everolimus (certican), temsirolimus (CCI-779) and zotarolimus (ABT-578).

In one embodiment, the hydrogel is coated onto said implantable medical device. In one embodiment, the implantable medical device is selected from the group consisting of vascular stents, shunts, vascular grafts, stent grafts, heart valves, catheters, pacemakers, pacemaker leads, bile duct stents and defibrillators.

In one embodiment, a method is described of forming a hydrogel comprising the steps of: a) choosing at least one macromer selected from the group consisting of Formula 1

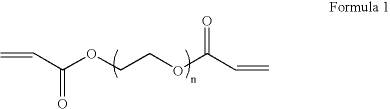

wherein n is between about 1 and about 500, Formula 2

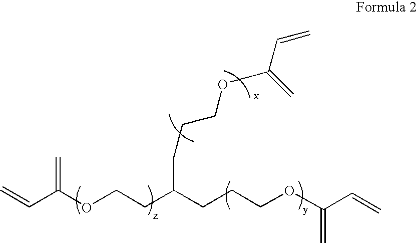

wherein x, y, and z are each independently between about 1 and about 500, or a combination thereof; b) choosing appropriate concentrations of said macromer(s); c) selecting an appropriate initiator; and d) reacting said macromers and said initiator, thereby forming a hydrogel. In one embodiment, the method further comprises the step of: e) coating at least a portion of a medical device, forming at least a portion of a medical device, or a combination of thereof with said hydrogel.

In one embodiment, the initiator is selected from the group consisting of light, Eosin Y, triethanolamine, a free radical, or combinations thereof. In one embodiment, the medical device is selected from the group consisting of vascular stents, shunts, vascular grafts, stent grafts, heart valves, catheters, pacemakers, pacemaker leads, bile duct stents and defibrillators.

### DEFINITION OF TERMS

Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, and other rapamycin hydroxyesters may be used in combination with the terpolymers of the present invention.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Cross-linking Agent: As used herein, “cross-linking agent” refers to a monomer that, when polymerized, covalently bonds one polymer chain to another.

Hydrogel: As used herein “hydrogel” refers to a water-containing polymer network.

Initiator: As used herein “initiator” refers to a molecule that initiates a polymerization reaction such as, but not limited to, an amino alcohol.

Macromer: As used herein “macromer” refers to a macromolecule, in particular a polymer that can be further polymerized or cross-linked.

Nonbiodegradable: As used herein “nonbiodegradable” refers to a polymeric composition that is biocompatible and not subject to being broken down in vivo through the action of normal biochemical pathways.

Photosensitive Molecule: As used herein “photosensitive molecule” refers to a molecule that becomes more reactive when exposed to light (photons).

### DETAILED DESCRIPTION OF THE INVENTION

Disclosed herein are polyethylene glycol (PEG) based hydrogels which can be coated on at least a portion of a medical device. The polyethylene glycol (PEG) based hydrogels described herein are also suitable for formation of at least a portion of a medical device. The hydrogels can be useful as they can be tailored to possess adhesive properties, making them useful in the field of implantable medical devices.

The hydrogels described herein can be nondegradable. The non-degradable nature of the hydrogels makes them ideal in that they are easy to store, transport, and sterilize. The hydrogels are formed from functionalized PEG based macromers. The functionalized group can be an acrylate group or another double bonded reactive group. Macromers suitable for forming the hydrogels can be linear or branched. Combinations of linear and branched macromers are also possible depending on the physical properties required.

A linear macromer can have the general structure of formula 1:

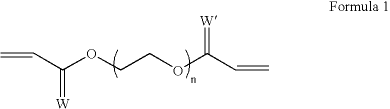

wherein W and W′ are each independently O or CH₂, n is between about 1 and about 500. In another embodiment, n is between about 1 and about 250; or about 1 and about 125; or about 1 and about 75.

A branched macromer can have the general structure of formula 2:

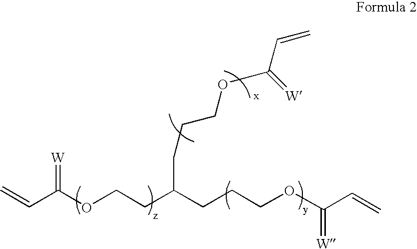

wherein W, W′ and W″ are each independently O or CH₂, x is between about 1 and about 500, or in other embodiments, x is between about 1 and about 250, or about 1 and about 125, or about 1 and about 75; y is between about 1 and about 500 or in other embodiments, y is between about 1 and about 250, or about 1 and about 125, or about 1 and about 75; and z is between about 1 and about 500 or in other embodiments, z is between about 1 and about 250, or about 1 and about 125, or about 1 and about 75. Each of x, y, and z can be changed independently of the others.

In one embodiment, linear and branched PEG based macromers can be used to form hydrogels as described herein. The physical properties of the hydrogel can be changed by adjusting the ratio of linear to branched macromers present in the hydrogel. In one embodiment, a ratio of 60:40 (linear to branched) can be used, in other embodiments, the ratio may be 50:50, or 25:75, or 75:25, or 5:95, or 95:5.

In order to tune, or modify, the polymers described herein, a variety of properties are considered including, but not limited to, glass transition temperature (T_{g}), connectivity, molecular weight and thermal properties.

The subject matter described herein also takes into account fine tuning, or modifying, the glass transition temperature (T_{g}) of the polymers. Drug elution from polymers depends on many factors including density, the drug to be eluted, molecular composition of the polymer and T_{g}. Higher T_{g}s, for example temperatures above 40° C., result in more brittle polymers while lower T_{g}s, e.g. lower than 40° C., result in more pliable and elastic polymers at higher temperatures. Drug elution is slow from polymers that have high T_{g}s while faster rates of drug elution are observed with polymers possessing low T_{g}s. In one embodiment, the T_{g} of the polymer is selected to be lower than 37° C.

In one embodiment, the polymers can be used to form and coat medical devices. Coating polymers having relatively high T_{g}s can result in medical devices with unsuitable drug eluting properties as well as unwanted brittleness. In the cases of polymer-coated vascular stents, a relatively low T_{g} in the coating polymer effects the deployment of the vascular stent. For example, polymer coatings with low T_{g}s are “sticky” and adhere to the balloon used to expand the vascular stent during deployment, causing problems with the deployment of the stent. Low T_{g} polymers, however, have beneficial features in that polymers having low T_{g}s are more elastic at a given temperature than polymers having higher T_{g}s as well as allowing the polymer to better adhere to surrounding tissues. Expanding and contracting a polymer-coated vascular stent mechanically stresses the coating. If the coating is too brittle, i.e. has a relatively high T_{g}, then fractures may result in the coating possibly rendering the coating inoperable. If the coating is elastic, i.e has a relatively low T_{g}, then the stresses experienced by the coating are less likely to mechanically alter the structural integrity of the coating. Therefore, the T_{g}s of the polymers can be fine tuned for appropriate coating applications by a combination of monomer composition and synthesis conditions. The polymers are engineered to have adjustable physical properties enabling the practitioner to choose the appropriate polymer for the function desired.

The polymerization process for the hydrogels described herein can be conducted in situ or in solution. The polymerization process can be conducted by any means known in the art including, but not limited to, photopolymerization, spontaneous polymerization, and temperature initiated radical polymerization wherein radicals are generated by heat and redox system polymerization wherein electrons are transferred thereby forming radicals.

In one embodiment, the hydrogel can be formed in solution by a method comprising photo-initiating the polymerization process by exposing at least one initiator, at least one photosensitive molecule and optionally at least one cross-linking agent to light of an appropriate wavelength to excite the photosensitive molecules, and then exposing macromers to reactive initiator and optionally reactive cross-linking agent, such that a hydrogel with improved adhesive properties is formed.

The in situ method can comprise applying a first mixture comprising at least one photosensitive molecule, at least one initiator, and optionally at least one cross-linking agent to a tissue surface. The tissue having the first mixture disposed thereon is exposed to light of an appropriate wavelength to excite the photosensitive molecule resulting in formation of an activated surface. A second mixture comprising at least one initiator, at least one cross-linking agent, and at least one polymerizable macromer as described herein is then applied to the same tissue surface or a second tissue surface. The second tissue surface my in some embodiments be a medical device or medical device coating. The tissues or surfaces to be adhered are then contacted with each other. Free radicals formed during the activation of the first mixture interact with the initiator and the cross-linking agent in the second mixture which, in turn, cause the polymerization and cross-linking of the macromer to form an adhesive hydrogel that adheres the two surfaces together.

Further, the photosensitive molecule can be evenly spread on the tissue to be treated along with an initiator molecule and optionally a cross-linking agent in a first mixture. The tissue is then exposed to light of the appropriate wavelength. The macromeric components of the hydrogel are then spread on the tissue along with an initiator molecule such as, but not limited to, methyldiethanolamine or triethanolamine, and a cross-linking agent such as, but not limited to, 2-vinyl pyrrolidinone. The photosensitive molecule, upon activation by light, forms a free radical and abstracts a proton from the initiator molecule which in turn attacks the cross-linking agent thereby cross-linking the macromer. The hydrogel formed in this manner provides adhesion between two tissue surfaces or between a tissue surface and a medical device or a tissue implant.

Photosensitive molecules suitable for forming the hydrogels described herein include, but are not limited to, photosensitive dyes, quinones, hydroquinones, poly alkenes, polyaromatic compounds, ketones, unsaturated ketones, peroxides, halides, Eosin Y, Eosin B, flourone, erythrosine, flourecsein, indian yellow, derivatives thereof, and/or combinations thereof. In one embodiment, the photosensitive molecule is Eosin Y. In another embodiment, the photosensitive molecule is Eosin B.

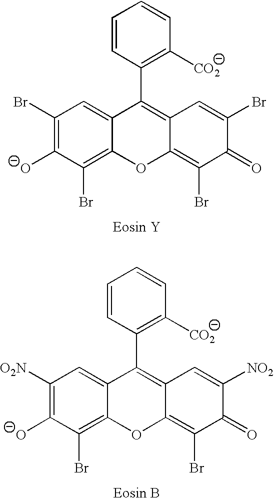

In another embodiment, the photosensitive molecule is fluorone.

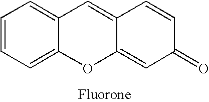

In another embodiment, the photosensitive molecule is erythrosine.

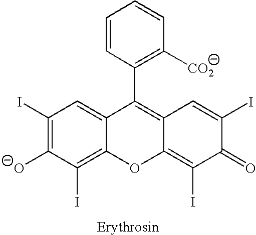

In another embodiment, the photosensitive molecule is fluorescein.

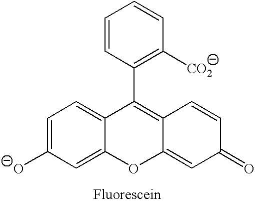

In another embodiment, the photosensitive molecule is indian yellow.

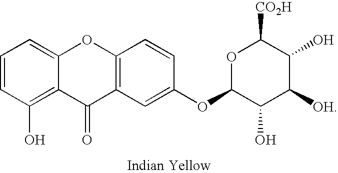

Each of the photosensitive molecules disclosed herein requires exposure to light of an appropriate excitation wavelength to activate the molecule. Each photosensitive molecule may have a different or similar excitation wavelength. For example, and not intended as a limitation, Eosin B is activated by light having wavelengths of 511-520 nm. In a further example also not intended as a limitation, Eosin Y is activated by light having a wavelength of approximately 490 nm. Excitation wavelengths of photosensitive molecules are well known to persons of ordinary skill in the art. Exemplary photosensitive molecules and the excitation wavelengths are described in U.S. Pat. No. 6,602,975 issued to Hubbell et al. which is incorporated by reference for all it contains regarding photosensitive molecules.

Initiator molecules suitable for forming the hydrogels include amino alcohols such as, but not limited to, methyldiethanolamine, triethanolamine (TEOA), thiols, C₁ to C₁₂ alkyls, C₃ to C₁₂ alkenyls, C₃ to C₁₂ alkynyls, C₆ to C₁₄ aryls, C₄ to C₁₂ heterocyclic alkyls, C₄ to C₁₂ heterocyclic alkenyls, C₄ to C₁₂ heterocyclic aryls, derivatives thereof, and/or combinations thereof.

Cross-linking agents suitable for forming the hydrogels include, but are not limited to, molecules with acetate, other double bonded groups, or other reactive groups. Exemplary cross-linking molecules include, but are not limited to, N-vinylpyrrolidone, polyethylene glycol derivatives, poly vinylpyrrolidinone, poly vinylpyrrolidinone derivatives, polyamides, polyurethanes, polysulfones, acrylates, derivatives thereof, and/or combinations thereof.

In another embodiment, the hydrogels may further comprise at least one bioactive agent. Bioactive agents suitable for incorporation within the hydrogels include, but are not limited to anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Further, exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein.

Furthermore, the adhesive hydrogels can also have cells or tissues disposed therein or thereon. Tissues and/or cells appropriate for the polymers described herein will be known by those skilled in the art and are dependent on the application of the hydrogel.

The hydrogels described herein can also be engineered for coating a medical device or actually being formed as a medical device. More specifically, the hydrogels can be used to coat or form implantable medical devices. The hydrogels can be applied to a medical devices by any appropriate means know to one skilled in the art. Such methods include, but are not limited to, spraying, dipping, injecting, brushing, or any of the methods described supra.

Exemplary medical devices include those in which tissue integration is desired, such as those that provide a sufficiently porous surface or can have a porous surface provided thereon including, but not limited to, joint implants (e.g., for hip or knee reconstruction), dental implants, soft tissue cosmetic prostheses (e.g., breast implants), wound dressings, vascular prostheses (e.g., vascular grafts and stents), and ophthalmic prostheses (e.g., intracorneal lenses). The hydrogels can be used in any suitable manner (e.g., to coat and/or fill voids within or upon the surface of the medical device).

The hydrogels described herein are also useful for biomedical applications such as, but not limited to, tissue adhesives, surgical adhesion prevention barriers, implantable wound dressings, scaffolds for cellular growth, tissue sealants, wound covering agents, controlled release adhesives, and barriers in preventing postoperative adhesions.

In addition, the hydrogels can be applied to a tissue site in any suitable manner know to those skilled in the art. Suitable examples include, but are not limited to spraying, dipping, injecting or brushing the first mixture and/or the second mixture on a substrate surface prior to cross-linking.

In some embodiments, the hydrogels can be used, for example, to provide adhesion between two tissue surfaces or between a tissue surface and the surface of a medical device. When the adhesive hydrogels provide adhesion between a tissue surface and the surface of a medical device, the medical device can be provided at the treatment site coated with at least one component of the hydrogel forming mixture. The hydrogel forming mixtures can be applied to the treatment site and/or the medical device before, during, or after implantation of the medical device at the treatment site. In addition, the hydrogels can be formed in situ using photo-polymerization techniques described herein.

In one embodiment, the hydrogel can be used to fill the spaces between a tissue implant and/or medical device (itself either tissue-based or non-tissue based) and adjacent tissue; this method is sometimes referred to as paving. Non-limiting exemplary tissue implants include both those obtained as transplants (e.g., autografts, allografts, or xenografts) and those provided by tissue engineering. Such tissue implants do not typically conform well to adjacent native tissue, thus leaving spaces into which undesirable fluids and cells can accumulate and produce adverse tissue responses. For example, when cultured cartilage is implanted into cartilage defects, synovial fluid and macrophages can enter the unfilled space and lead to fibrous tissue formation, which prevents integration of the implanted cartilage with the native cartilage. Other cultured tissues that are implanted into tissue defects, and that would benefit from the present macromer system applied as a grout include, but are not limited to, skin, bone, ligaments, blood vessels, and heart valves.

Hydrogels and macromers as described herein are nondegradable. Nondegradable hydrogels and macromers have the advantage of being easy to store, transport and sterilize. Hydrogels and the macromers as described herein can be stored at room temperature with some protection such as nitrogen protect or the addition of inhibitor to the hydrogel. Conventional sterilization methods can be incorporated with the hydrogels described herein, such as but not limited to gamma irradiation.

### EXAMPLE 1

### PEG Macromer Synthesis

PEG3400 (60 g) was dissolved in anhydrous dichloromethane under N₂ protection. Triethylene amine (9.1 ml) was added into the solution slowly. Acrylic chloride (5.5 ml) was added dropwise into the solution upon stirring. The reaction was continued for 3 days and the polymer was precipitated into ethyl ether.

### EXAMPLE 2

### Partial Coating on a Stent Graft

In one embodiment, the hydrogel of the present invention can be coated onto a vascular stent graft. The hydrogel can be applied to a predetermined portion of the stent. Further, the hydrogel can be loaded with growth hormone in order to enhance tissue growth upon the graft. For example, the hydrogel can be coated around the neck of an abdominal aortic aneurysm stent graft in order to enhance the anchoring of the graft. Upon implantation into the abdominal aorta of a patient, the adhesive properties the hydrogel can allow for better anchoring of the graft.

### EXAMPLE 3

### Paving into a Vessel

In one embodiment, the hydrogel can paved around the outside of a catheter and inside of the superficial femoral artery (SFA) thereby forming a flexible stent inside the vessel. The hydrogel can be loaded with an anti-proliferative drug such as zotarolimus which will aid in the acceptance of the implant. The formation of the hydrogel from functionalized macromers can be by method of in situ formation using an appropriate wavelength of light to initiate cross-linking of the hydrogel.

### EXAMPLE 4

### Formation of a Cross-Linked Linear Hydrogel

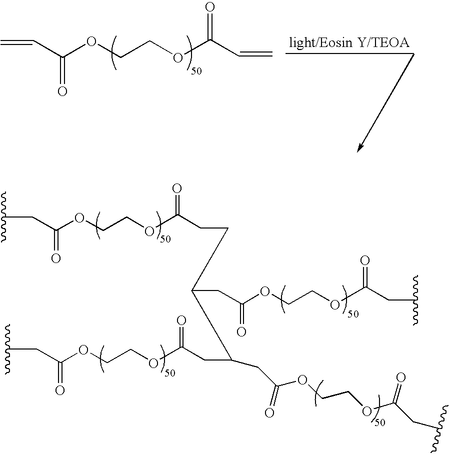

In the present example, 200 grams of polyethylene glycol diacrylate is mixed with 5 grams of Eosin Y and 5 grams of triethanolamine (TEOA). The solution is subjected to light of about 490 nm, exciting the Eosin Y. The radicalized Eosin Y thereby drives the polymerization of the hydrogel.

Unless otherwise indicated, all numbers expressing quantities of ingredients, properties such as molecular weight, reaction conditions, and so forth used in this specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on the described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A polymeric material formed from macromers of a general formula 1: 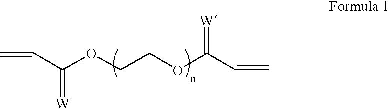
wherein W, W′ are each independently O or CH₂, n is between about 1 and about 500, macromers of a general formula 2: 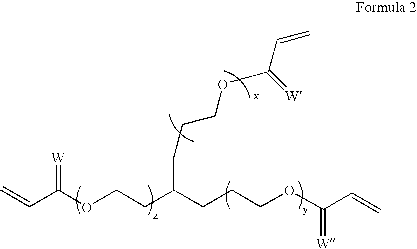
wherein W, W′ and W″ are each independently O or CH₂, x, y, and z are each independently between about 1 and about 500, or combinations thereof, and wherein at least two macromers are cross-linked thereby forming a hydrogel.

2. A polymeric material according to claim 1, wherein said hydrogel is non-degradable.

3. A polymeric material according to claim 1, wherein at least one of n, x, y, or z is the following:
n is between 1 and 250;
x is between 1 and 250;
y is between 1 and 250; and
z is between 1 and 250.

4. A polymeric material according to claim 1, wherein said hydrogel further comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

5. A polymeric material according to claim 1, wherein said hydrogel further comprises at least one bioactive agent selected from the group consisting of sirolimus (rapamycin), tacrolimus (FK506), everolimus (certican), temsirolimus (CCI-779) and zotarolimus (ABT-578).

6. A polymeric material according to claim 1, wherein said hydrogel comprises any ratio of formula 1: formula 2.

7. An implantable medical device comprising a polymeric material, said polymeric material formed from macromers of a general formula 1: 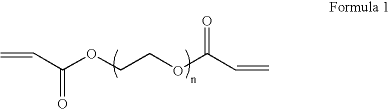
wherein n is between about 1 and about 500, macromers of a general formula 2: 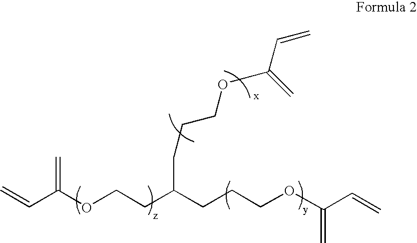
wherein x, y, and z are each independently between about 1 and about 500, or combinations thereof, and wherein at least two macromers are cross-linked thereby forming a hydrogel.

8. An implantable medical device according to claim 7, wherein said hydrogel is non-degradable.

9. An implantable medical device according to claim 7, wherein said hydrogel comprises at least one of n, x, y, or z with the following:
n is between 1 and 250;
x is between 1 and 250;
y is between 1 and 250; and
z is between 1 and 250.

10. An implantable medical device according to claim 7, wherein said polymeric material further comprises at least one bioactive agent selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

11. An implantable medical device according to claim 7, wherein said hydrogel further comprises at least one bioactive agent selected from the group consisting of sirolimus (rapamycin), tacrolimus (FK506), everolimus (certican), temsirolimus (CCI-779) and zotarolimus (ABT-578).

12. An implantable medical device according to claim 7, wherein said hydrogel comprises any ratio of formula 1: formula 2.

13. An implantable medical device according to claim 7, wherein said hydrogel is coated onto said implantable medical device.

14. An implantable medical device according to claim 7, wherein said implantable medical device is selected from the group consisting of vascular stents, shunts, vascular grafts, stent grafts, heart valves, catheters, pacemakers, pacemaker leads, bile duct stents and defibrillators.

15. A method of forming a hydrogel comprising the steps of:
a) choosing at least one macromer selected from the group consisting of Formula 1 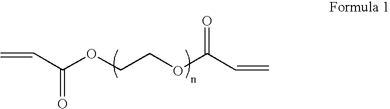
wherein n is between about 1 and about 500, Formula 2 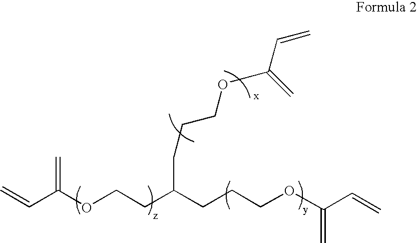
wherein x, y, and z are each independently between about 1 and about 500, or a combination thereof;
b) choosing appropriate concentrations of said macromer(s);
c) selecting an appropriate initiator; and
d) reacting said macromers and said initiator, thereby forming a hydrogel.

16. A method according to claim 15, wherein the method further comprises the step of:
e) coating at least a portion of a medical device, forming at least a portion of a medical device, or a combination of thereof with said hydrogel.

17. A method according to claim 15, wherein said initiator is selected from the group consisting of light, Eosin Y, triethanolamine, a free radical, or combinations thereof.

18. A method according to claim 15, wherein said medical device is selected from the group consisting of vascular stents, shunts, vascular grafts, stent grafts, heart valves, catheters, pacemakers, pacemaker leads, bile duct stents and defibrillators.
